(19) United States
(12) Patent Application Publication
(10) Pub. No.: US 2016/0082055 A1
(43) Pub. Date: Mar. 24, 2016

(54) COMPOSITION COMPRISING A COMBINATION OF AN ELDER EXTRACT AND A STRAIN OF LACTOBACILLUS RHAMNOSUS

(71) Applicant: PIERRE FABRE MEDICAMENT, Boulogne-Billancourt (FR)

(72) Inventors: Christian Jacques LATGE, Balma (FR); Christine LIBON, Castanet-Tolosan (FR)

(73) Assignee: PIERRE FABRE MEDICAMENT, Boulogne-Billancourt (FR)

(21) Appl. No.: 14/783,181

(22) PCT Filed: Apr. 8, 2014
(86) PCT No.: PCT/EP2014/057082

§ 371 Date: Oct. 8, 2015

(30) Foreign Application Priority Data
Apr. 9, 2013 (FR) 1353193

(51) Int. Cl. A61K 35/747; A61K 36/185

(52) U.S. Cl. CPC A61K 35/747; A61K 36/185

(57) ABSTRACT

The present invention concerns a combination of an elder (Sambucus nigra) extract and a strain of Lactobacillus rhamnosus, intended to stimulate immunity and/or strengthen the immune defences and/or promote an anti-infection and/or anti-inflammatory immune response and/or help maintain vitality.

The present invention concerns a composition comprising a combination of an elder extract and at least one strain of Lactobacillus rhamnosus, intended to stimulate immunity and/or promote an anti-infection and/or anti-inflammatory response.

The elder or Sambucus nigra L. belongs to the family of Adoxaceae. The genus Sambucus comprises 25 species distributed throughout the world in temperate regions. The black elder, S. nigra ssp. nigra, is widely grown in Europe, especially in Denmark, in Italy and in Austria, in the northern region of Africa and in the western part of Asia. The Canadian elder, S. nigra ssp. canadensis, is native to the north east of North America and grown in Oregon, in Pennsylvania and in Kansas, despite a selection made in Canada.

The different organs of the elder: barks, roots, stem, flowers, leaves and fruits are traditionally used in fields as diverse as medicine, the food industry and the manufacture of tools and toys. The most important industrial productions nowadays concern the production of extracts of elder berries destined for nutraceutical markets and the colorants industry.

Certain publications report the effect of an extract of elder berries on the production of cytokines (Barak et al., European Cytokine network, Vol 12(2), 290-296, 2001). The effect has especially already been demonstrated of an elder extract on the alleviation of symptoms linked to a flu infection due to Influenza viruses (A and/or B and/or C) as well as its antiviral effect on type 1 herpes simplex viruses, respiratory syncytial virus and Influenza viruses (Zakay-Rones et al., The Journal of International Medical Research, vol 32, 132-140, 2004).

Numerous scientific studies have demonstrated the beneficial effects on health of certain microorganisms present in fermented foods, especially dairy products. These microorganisms are commonly called “probiotics”. According to the definition generally accepted at the present time, probiotics are: living microorganisms, which when they are consumed in adequate quantities, have a beneficial effect on the health of the host (WHO report on the evaluation of the health and nutritional properties of probiotics in foods, including powdered milk containing living lactic bacteria, Cordoba, Argentina, 1-4 Oct. 2001).

It has been shown in the patent applications WO96/20607, EP0794707, EP1283714 and FR292912657 that the consumption of food products containing probiotic bacteria can produce favourable effects on health, especially through rebalancing of the intestinal flora, the improvement of resistance to infections, and the modulation of the immune response.

The probiotic microorganisms used in the human diet are generally lactic bacteria, mainly belonging to the genuses Lactobacillus and Bifidobacterium and especially to the species Lactobacillus paracasei.

Lactobacillus is a genus of gram positive bacteria, immobile, of variable forms and dimensions, optionally anaerobic. It is thus named because most of its members convert lactose and other simple sugars into lactic acid. In humans, lactobacilli are hosts that are very widespread as commensals and generally useful, or even necessary. They constitute an important element of the intestinal flora.

Lactobacillus rhamnosus is a bacterium that was initially considered as a sub-species of Lactobacillus casei, but genetic research has proved that it is a distinct species. Lactobacillus rhamnosus is normally found in the stomach and in the digestive tract. This bacterium has properties that are beneficial to the intestinal tract, but also for the immune system in particular in combatting against pathogenic agents of the intestinal and urinary tracts.

Probiotics can exert direct effects on the chyme, the flora, referred to as luminal effects, or at the level of enterocytes or GALT immunocompetent cells, then referred to as skin effects. They can also have indirect effects linked to modifications of the ecosystem or the local immune system.

Certain probiotics have a capacity of adhering to the digestive epithelium. This property may constitute and ecological advantage favouring the chances of close interrelations with the enterocyte epithelium and the local immune system. A study has shown that an adherent strain of Lactobacillus rhamnosus could colonise in a prolonged manner the jejunal and/or rectal mucosa in some subjects (Alander et al., Letters in Applied Microbiology, 24 (5), vol 361-364, 1997).

More and more numerous works demonstrate the biological effects of multiple strains, it is important to note that these effects appear to be strain-dependent.

The patent application US 2006/0233895 describes a composition comprising an elder extract which may potentially be combined with a probiotic such as Lactobacillus casei. Nevertheless, this composition necessarily contains a mixture of Uncaria tomentosa, Pau d′Arco, Scrutellaria baicalensis and Artemisin. Moreover, this composition is not intended to stimulate immunity but is used to treat Lyme's disease which is caused by a bacterial infection and which is thus very different to infections by viruses of the influenza type.

The patent application WO 2010/043696 discloses a composition comprising a combination of a black elder extract and a strain of Lactobacillus paracasei, Lactobacillus casei, Lactobacillus bulgaricus, or Streptococcus thermophilus intended to stimulate immunity. In this application, a synergy between elder and Lactobacillus paracasei is demonstrated for the production of IL-10 and interferon-gamma. Nevertheless the limit of the inflammatory reaction is not known. Data on other cytokines and chemokines of the CXCL10 type would take away the risk of a too important increase in this inflammatory response.

The present invention thus concerns a combination comprising an elder extract and at least one strain of Lactobacillus rhamnosus.

Advantageously, such a combination is a synergetic combination.

The present invention also concerns a composition comprising such a combination and advantageously a composition comprising an elder extract and at least one strain of Lactobacillus rhamnosus.

The present invention also concerns a composition comprising an elder extract and at least one strain of Lactobacillus rhamnosus and moreover vitamins and/or mineral salts.

In a particular embodiment, the composition according to the invention may be prepared by the method comprising the following steps:
preparation of the combination;
addition of the combination into the composition according to the present invention.

In this case, the combination is thus prepared before its addition to the composition. Nevertheless it is also possible to prepare a composition according to the present invention by addition of each of the constituents of the combination in a separate manner into the composition, that is to say without preparing the combination beforehand.

Preferentially, the composition according to the invention is intended for administration by oral route.

Advantageously the composition according to the present invention is a food, a food supplement, a medicine or an OTC (Over The Counter) product.

Advantageously, the elder extract according to the present invention is obtained from elder berries and/or flowers, and preferentially from elder berries. In an advantageous manner, the elder extract according to the present invention is a water soluble extract.

The elder extract used within the scope of the present invention may be characterised by on the one hand its anthocyanin content and on the other hand by large molecules such as proteins (in particular lectins). Anthocyanins are natural pigments of leaves, petals and fruits, situated in the vacuoles of cells, soluble in water, ranging from the orangey red to purple blue in the visible spectrum.

Advantageously, the elder extract comprises anthocyanins, in an advantageous manner of the family of pelargonidins and cyanidins, in a quantity comprised between around 0.5% and around 25%, in an advantageous manner between around 3% and around 25% and in an even more advantageous manner between around 8% and around 16% by weight, compared to the dry matter of the extract. Preferably, the anthocyanin content is equal to around 12% by weight, compared to the dry matter of the extract. These contents are expressed in cyanidin-3-glucoside according to the HPLC method described by Wu et al., (J. Agric. Food Chem. 52 (26), 7846-7856, 2004).

Advantageously, the elder extract comprises proteins in a quantity comprised between 2 and 10% by weight, expressed compared to the dry matter (N×6.25 according to the Kjeldahl method: Protéines végétales, Coord. B. Godon, Collection Sciences et techniques agro-alimentaires, Technique et Documentation Lavoisier, Paris, 1985).

Within the scope of the present invention, the elder extract is commercially available or may be obtained by a method comprising the following steps:

The fresh or frozen elder berries may undergo a pressing in order to obtain a juice after filtration. It may undergo a step of enzymatic hydrolysis (pectinase or mixture of pectin methylesterase and polygalacturonase) in order to clarify it before filtration (Girard and Fukumoto, Crit. Rev. Food Sci. Nutr. 40(2), 91-157, 2000). An aqueous extraction of the dried and ground berries may also be carried out, followed by a solid/liquid separation by filtration. The extracts thereby obtained may be used as such or concentrated, or dried in the form of powder.

The anthocyanin content, at this stage, is comprised between 0.5% and 3% by weight compared to the dry matter of the extract, and advantageously equal to around 1% (HPLC assay, expressed in cyanidin-3-glucoside).

The elder extract may be enriched, in particular in anthocyanins and/or in high molecular weight molecules (proteins, polyphenols, polysaccharides). To do so, several methods well known to those skilled in the art are possible:
Ultrafiltration: step of diafiltration on the juice diluted beforehand then over-concentration on organic or mineral membrane with a cut-off threshold comprised between 1 and 20 kDa, and preferentially comprised between 3 and 10 kDa (Girard and Fukumoto, Crit. Rev. Food Sci. Nutr. 40(2), 91-157, 2000).
Passage on Amberlite XAD type absorbent polymeric resin column.
Extraction with water-alcohol (mixture in all proportions of water and C1 to C4 alcohol) or aqueous solutions at acid pH (Bronnum-Hansen and Flink, Int. J. Food Sci. Tech. 21(2), 605-614, 1986; Lee and Wrolstad, J. Food Sci. 69(7), 564-573, 2004). This extraction may advantageously be carried out directly on dried and ground berries or on the purée produced from fresh berries.

The extract thereby obtained may be concentrated thermally (at a temperature not exceeding 50° C. and under vacuum) in order to increase the Brix degrees and stabilise vis-à-vis microbiological contaminations. It may also be dried alone or on a support (for example maltodextrin, lactose, etc.).

The enriched juice obtained lies preferentially between 40 and 60° Brix, between 3 and 25% of anthocyanins and between 5 and 10% of proteins (Kjeldahl method, N×6.25) expressed by weight compared to the dry matter.

Advantageously, the quantity of dry extract of elder per unit dose present in the composition, according to the invention, is comprised between around 10 mg and around 1 g. More advantageously, the composition comprises between around 20 mg and around 200 mg of dry extract of elder per unit dose and in a more advantageous manner, between around 20 and 100 mg of dry extract of elder per unit dose, and in an even more advantageous manner between 40 mg and 80 mg of dry extract of elder per unit dose, and in another just as advantageous manner between 20 and 60 mg of dry extract of elder per unit dose.

Within the scope of the present invention, the term “unit dose” represents the quantity of composition according to the invention administered at a single time. Advantageously a unit dose according to the present invention may correspond for example to a yoghurt of 100 or 125 ml, to a capsule of conventional size or to a 2 g tablet. Thus, whatever the weight of the composition, the quantity of dry extract of elder and/or strain of Lactobacillus rhamnosus per unit dose remains constant.

A strain of Lactobacillus rhamnosus quite particularly suitable within the scope of the present invention is the strain Lactobacillus rhamnosus GG (ATCC 53103). This strain of Lactobacillus rhamnosus is a strain isolated in 1983 in the intestinal tract in healthy humans. This strain is the subject matter of the U.S. Pat. No. 4,839,281. The strain Lactobacillus rhamnosus GG has been shown to be beneficial in the prevention of different types of diarrhoea in adults and in children (Guandalini et al., J. Pediatr. Gastroenterol. Nutr. 30(1), 54-60, 2000; Osterlund et al., Br. J. Cancer 97, 1028-1034, 2007). It has also been reported that the strain Lactobacillus rhamnosus GG was at the origin of a reduction in the risk of infections of the respiratory tract in children (Hojsak et al., Clin. Nutr. 29(3), 312-316, 2010).

Lactobacillus rhamnosus is also a considerable aid for the immune system, in particular in combatting pathogenic agents of the intestinal and urinary tracts. Lactobacillus rhamnosus attaches itself to the mucous of the intestine, where it encourages the growth of good bacteria which help digestion. Lactobacillus rhamnosus is a bacterium which helps to eliminate and to prevent the growth of harmful bacteria in the intestines.

Advantageously, the composition will include 1.10⁷ to 1.10¹¹ CFU of Lactobacillus rhamnosus per unit dose, preferentially 1.10⁸ to 1.10⁹ CFU per unit dose and in an even more preferred manner 5.10⁸ CFU per unit dose.

In a particular embodiment of the invention, the composition comprises, per unit dose,
from 10 mg to 1 g of dry extract of elder, preferentially from 20 mg to 200 mg, advantageously from 20 mg to 100 mg and more preferentially from 40 mg to 80 mg; and in another just as advantageous manner from 20 to 60 mg.
from 1.10⁷ to 1.10¹¹ of Lactobacillus rhamnosus, preferentially from 1.10⁸ to 1.10⁹ CFU per unit dose.

In a particular embodiment of the invention, the composition comprises, per unit dose,
from 2 mg to 200 mg of anthocyanins of elder, preferentially from 2 mg to 100 mg and more preferentially from 2 mg to 20 mg, and in just as advantageous a manner from 20 mg to 50 mg.
from 2 mg to 100 mg of proteins of elder, preferentially from 10 mg to 20 mg; and
from 1.10⁷ to 1.10¹¹ of Lactobacillus rhamnosus, preferentially from 1.10⁸ to 1.10⁹ CFU per unit dose.

In an advantageous manner a synergy effect is then observed.

The combination of an elder extract and a strain of Lactobacillus rhamnosus according to the invention has shown good capacities to induce and/or stimulate a non-specific immune response thereby demonstrating its interest in the food and/or pharmaceutical fields.

The composition according to the present invention may advantageously come in all the dosage forms normally used in the food and/or pharmaceutical fields for administration by oral or sublingual route.

In the case of a food, it may be especially fresh dairy products, fermented dairy products, yoghurts, fermented milks, infant milks, powders, pastilles, vegetable juices, drinks and mixtures thereof, advantageously in the group constituted of fresh dairy products, fruit and/or vegetable juices or fruit compotes.

In an advantageous manner, the fruit is chosen from the group constituted of apple, orange, red fruits, strawberry, peach, apricot, prune, raspberry, blackberry, red current, lemon, grapefruit, banana, pineapple, kiwi, pear, cherry, coconut, passion fruits, mango, fig, rhubarb, melon, exotic fruits, lychee, raisins, bilberry or mixtures thereof.

“Food supplement” is taken to mean within the sense of the present invention a foodstuff, the aim of which is to complete the normal diet and which constitutes a concentrated source of nutrients or other substances having a nutritional or physiological effect alone or combined.

Advantageously, the food supplement may comprise vitamins and/or minerals and/or trace elements.

Among these vitamins may in particular be cited the vitamins A, C, D and E and vitamins of the B group, especially, B1, B2, B3, B5, B6, B8, B9 and B12.

Vitamin A (retinol or carotenoids), vitamin E and vitamin C are the 3 major antioxidant vitamins. Vitamin A is present uniquely in foods of animal origin. Nevertheless, certain plants contain A provitamins, that is to say substances which the body is capable of transforming into vitamin A. It is indispensable for vision and for the growth of the bronchial tubes, intestines or the skin. Vitamin A is also involved in immune mechanisms. Antioxidant substances oppose the effects of free radicals by moderating their damage, restoring lesions undergone by cells, eliminating wastes, impeding the phenomenon of oxidation. When free radicals are in excess in the body, they can promote cell ageing. These antioxidant vitamins have the capability of controlling the production of free radicals derived from the metabolism of oxygen, and the over-production of which is responsible for very many disorders, especially at the cardio-vascular level, immunity, cancer, skin, accelerated ageing, neuro-degenerative diseases, etc. In particular, vitamin C is a well-known antioxidant, soluble in water. Humans depend on external sources of vitamin C to satisfy their vitamin C requirements. Thus, ascorbic acid, sodium ascorbate, or mixtures thereof are a source of vitamin C. Vitamin C is necessary for the synthesis of blood vessels and muscles. It promotes the absorption of iron present in foods and intervenes in several hormonal mechanisms. It also plays a role in the elimination of toxic substances. Vitamin C deficiency can reduce resistance to infections. Vitamin E is also a well-known antioxidant. Vitamin E can operate in synergy with vitamin C in order to protect the vital cell function against normal oxidants. Alpha-tocopheryl acetate, trimethyl-tocopheryl acetate and/or the succinate of vitamin E are sources of vitamin E. Vitamin E has a particularly important protective effect vis-vis the cells of the body. It plays an important role in procreation mechanisms and is involved in the synthesis of red blood cells.

Vitamin D is designated as a lipid soluble vitamin, even though it is above all a hormone synthesized in the human body from a derivative of cholesterol under the action of the UVB rays of light. It exists in 2 forms, D2 (ergocalciferol) or D3 (cholecalciferol). These 2 molecules are 9,10-secosteroids. Vitamin D is involved in the absorption of calcium and phosphorous by the intestines, as well as in their reabsorption by the kidneys, it is a true hormone. On the other hand, it influences more than 200 genes, which explains its importance, unexpected until recently, in numerous illnesses of which arthritis, skin disorders related to psoriasis, diabetes, certain cancers and even dementia.

The B vitamins form a group of water soluble vitamins which play an important role in the metabolism of cells, among which may be found:
Vitamin B1 or thiamine, which is the cofactor of the complex pyruvate decarboxylase;
Vitamin B2 or riboflavin which is the precursor of flavin adenine dinucleotide;
Vitamin B3 (PP) or nicotinamide which is the precursor of nicotinamide adenine dinucleotide;
Vitamin B5 or panthothenic acid which is the precursor and constituent of coenzyme A;
Vitamin B6 or pyridoxine which is the coenzyme of reactions linked to the metabolism of amino acids and proteins;
Vitamin B8 (H) or biotin which is the coenzyme of metabolism reactions of fatty acids, carbohydrates and amino acids;
Vitamin B9 or folic acid and vitamin B12 or cyanocobalamin which are cofactors with S-adenosylmethionine.

The B vitamins often function together to the benefit of the health of the body. They increase metabolism, help in maintaining a healthy skin and muscles in good health, improve the immune system, improve the nervous system and delay cognitive disorders of Alzheimer's disease, promote the growth of cells as well as their division, they combat the symptoms caused by an excess of stress.

Advantageously, the minerals are chosen from iron, zinc, selenium, chromium, manganese, molybdenum and mixtures thereof. Mineral salts are substances derived from rocks which enter into the composition of organisms and which are present in animal and plant feed. They come in ionic form, the major mineral elements are distinguished, such as calcium, iron, magnesium, phosphorous, potassium, sodium, sulphur and trace elements such as aluminium, arsenic, boron, chlorine, chromium, cobalt, copper, fluorine, iodine, manganese, molybdenum, nickel, lead, silicon, selenium, vanadium, zinc.

Iron is one of the mineral salts essential to the correct functioning of the body. It has a fundamental role in the constitution of the haemoglobin contained in red blood cells, in the constitution of myoglobin contained in the muscles and in that of numerous enzymes indispensable for the functioning of the body. Iron deficiency is frequent throughout the world, it results in anaemia, accompanied by a reduction in physical and intellectual capacity, a reduction in resistance to infections.

Zinc plays an antioxidant role as cofactor of enzymes which participate directly in the defence against oxidants. Zinc oxide, zinc gluconate, zinc citrate, zinc acetate, zinc chloride, zinc lactate or zinc sulphate may be used alone or in a mixture in the compositions according to the present invention. Advantageously it is zinc gluconate.

Selenium also plays an antioxidant role as cofactor of enzymes, especially glutathione peroxidase, which participates directly in the defence against oxidants.

Chromium is a trace element which is involved in the metabolism of carbohydrates, it promotes the action of insulin. It is also involved in the metabolism of lipids and in that of cholesterol, it contributes to reducing its concentration. Chromium also plays its role in the metabolism of nucleic acids.

Manganese is a trace element indispensable to vitamin B1 efficiency. It also intervenes in certain metalloproteins such as superoxide dismutase.

It acts as cofactor of numerous enzymes. It is also an essential metal for the synthesis of enzymes participating in combatting against oxidative stress and which prevents damage caused by free radicals.

Molybdenum is involved at numerous levels as constituent of certain enzymes, especially xanthine oxidase.

It plays a role in bone growth and the structure of teeth. It has a favourable action on the metabolism of iron. Molybdenum also participates in the detoxification of the body and it is involved in the synthesis of uric acid.

Optional additional active ingredients may be added according to the invention. For example, the coenzyme Q10 and/or a Ginseng extract. The coenzyme Q10 or ubiquinone is a substance similar to a vitamin which is vital for the functioning of the human body. The coenzyme Q10 is naturally present in all human cells and assures the production of body energy in the mitochondria. Around 95% of corporal energy requirements are transformed by means of the coenzyme Q10. This substance cannot be replaced by any other substance. If through dietary supplement the intake of coenzyme Q10 is increased, the capacity to exercise is strengthened. The lipidic metabolism is more efficient, as is the maximum consumption in oxygen and duration of effort. The reduction in the level of coenzyme Q10 is linked to ageing, but also to stress.

Ginseng is the medicinal plant which has the greatest reputation in Asia. Ginseng is above all a stimulant of the nervous, physical and intellectual system, and enhances physical resistance. It is a stimulant, vasomotor which has a fundamental action on the body, it enables better resistance to various stresses.

The composition according to the present invention may be administered by oral route or any other pharmaceutical administration route.

The compositions according to the present invention may be formulated for administration to mammals, including humans. These compositions are produced so as to be able to be administered by oral, sublingual, subcutaneous, intramuscular, intravenous, transdermal, local or rectal route. In this case, the active ingredients may be administered in unit administration forms, mixed with conventional pharmaceutical supports, to animals or to human beings.

In the case of a food supplement or a medicine, the following dosage forms may be envisaged: capsules, tablets to swallow, tablets to chew, effervescent tablets, pastilles, pills, powders, granules, oral solutions or suspensions and sublingual and buccal administration forms, subcutaneous, topical, intramuscular, intravenous, intranasal or intraocular and rectal administration forms. The preferential dosage form is the capsule.

When a solid composition is prepared in tablet form, the active ingredients are mixed with a pharmaceutical vehicle such as gelatine, starch, lactose, magnesium stearate, talc, gum arabic, silica or analogues. It is possible to coat the tablets with sucrose or other suitable materials or instead they may be treated such that they have an extended or delayed activity and that they release in a continuous manner a predetermined quantity of active ingredient.

A preparation in capsules is obtained by mixing the active ingredients with a diluent (optional step) and by pouring the mixture obtained into soft or hard capsules.

A preparation in the form of syrup or elixir may contain the active ingredients together with a sweetener, a flavouring agent and a suitable colorant.

Powders or granules dispersible in water may contain active ingredients mixed with suspension agents, as well as flavouring agents or sweeteners.

For rectal administration, resort is made to suppositories which are prepared with binders that melt at rectal temperature, for example cacao butter or polyethylene glycols.

For parenteral (intravenous, intramuscular etc.), intranasal or intraocular administration, use is made of aqueous suspensions, isotonic saline solutions or sterile and injectable solutions which contain dispersion agents and/or pharmacologically compatible wetting agents.

The active ingredients may also be formulated in the form of microcapsules, potentially with one or more additive supports.

Advantageously, the composition according to the present invention is intended for administration by oral route.

Another object of the present invention is a composition according to the invention for its use as medicine.

Another object of the present invention is a composition according to the present invention for its use to stimulate immunity and/or strengthen the immune defences and/or promote an anti-infection and/or anti-inflammatory response and/or in maintaining vitality.

It is understood that the use relative to maintaining vitality is founded on claims for the vitamins B2, B5, B6, B12, C and iron. These are health claims authorised by the European Food Safety Authority (EFSA) on these nutrients. They are based on scientific data which measure the metabolic production of energy values via the use of carbohydrates, lipids or proteins. This also impacts the synthesis of hormones which act on the metabolism, such as steroidal hormones.

This use is thus supported by the following references:
EFSA Journal 2009; 7(9): 1215 Scientific opinion on the substantiation of health claims related to iron and formation of red blood cells and haemoglobin (ID 249, ID 1589), function of the immune system (ID 252, ID 259), cognitive function (ID 253) and cell division (ID 368) pursuant to Article 13(1) of regulation (EC) n° 1924/2006.
Huskisson E., Maggini S., Ruf M., Journal of International Medical Research 2007, 25: 277-289. The role of vitamins and minerals in energy metabolism and well-being.
Institute of Medicine (1998), National Academy press, Washington, D.C. (592 pages). Dietary reference intakes for Thiamine, Riboflavin, Niacin, Vitamin B6, Folate, Vitamin B12, Pantothenic acid, Biotin, and Choline.
A report of the standing committee on the scientific evaluation of dietary reference intakes and its panel of folate, other B vitamins, and choline and subcommittee on upper reference levels of nutrients food and nutrition board institute of medicine.

Another object of the present invention is a composition for its use in the treatment and/or the prevention of respiratory and/or intestinal infections.

Another object of the present invention is composition for its use in the treatment and/or the prevention of the symptoms caused especially by an infection by type 1 herpes simplex virus, the respiratory syncytial virus and Influenza viruses (A and/or B and/or C) and parainfluenza, advantageously the symptoms of flu-like states, especially caused by Influenza viruses (A and/or B and/or C).

The composition according to the invention is particularly suitable for the treatment and/or the prevention of the symptoms of flu-like states caused by an infection by Influenza viruses (Orthomyxovirus, Influenzae A, B or C). These symptoms include colds, rhinitis, coughs, inflammations of the nasal mucous (nasal discharge, nasal obstruction, sneezing attacks), sore throat (different manifestations: tingling, difficulty swallowing), fever, headaches, aches, fatigues.

These “winter ailments” are associated with the weakening of our immune defences.

Another object of the present invention is the use of a composition for the preparation of a food, a food supplement or a medicine.

Another object of the present invention is the use of the composition for the preparation of a medicine intended for the treatment and/or the prevention of respiratory and/or intestinal infections.

Another object of the present invention is the use of the composition for the preparation of a medicine intended for the treatment and/or for the prevention of the symptoms of an infection by type 1 herpes simplex virus, the respiratory syncytial virus and Influenza viruses (A and/or B and/or C) and parainfluenza, advantageously the symptoms of flu-like states, especially caused by Influenza viruses (A and/or B and/or C).

Another object of the present invention is the use of the composition for the preparation of a medicine for its use to stimulate immunity and/or promote an anti-infection and/or anti-inflammatory response and/or help maintain vitality.

It is finally another object of the present invention to provide a method of strengthening the immune defences which consists in an administration by oral route of a composition based on a combination of an elder extract and at least one strain of Lactobacillus rhamnosus.

The composition according to the invention is suitable for all ages of life. It is particularly suitable for very young children. “Very young children” is taken to mean within the sense of the present invention a child from 6 months up to 3 years old. The composition according to the invention is quite particularly suited for “juniors”, where “junior” is taken to mean within the sense of the present invention a child from 3 years up to 17 years old. The composition according to the invention is also particularly suitable for adults, where “adult” is taken to mean within the sense of the present invention a person from 18 years to 60 years old. But the composition according to the invention is also quite particularly suitable for “seniors”, where “senior” is taken to means within the sense of the present invention a person at least 60 years old.

The invention could be better understood by means of the non-limiting examples that follow and which constitute particular embodiments of the compositions according to the invention.

### EXAMPLE 1

### Preparation of an Elder Extract Used within the Scope of the Present Invention

The elder extract is obtained from juice of elder berries enriched in anthocyanins and dried on maltodextrin. The enrichment was carried out by membrane filtration process according to the following method: 26 litres of clarified elder juice are diluted with 284 litres of water (11 volumes of water for 1 volume of juice). The 310 litres thereby obtained are stirred and heated to 45° C. then diafiltered on a first 5 kDa membrane by adding 7 volumes of water per volume of juice (i.e. 181 L). The dialysis permeate is then concentrated by ultrafiltration on 5 kDa membrane (pilot membrane of 5/10 kDa of 5.8 m² unit surface). The retentate from the ultrafiltration is thermally concentrated under vacuum at a temperature not exceeding 50° C., then atomised onto a maltodextrin support such that the quantity of maltodextrin is less than or equal to 30% (w/w).

The characteristics of the extract thereby obtained are:
Anthocyanin content: 10-14% by weight compared to the dry matter of the extract (expressed in cyanidine-3-glucoside according to the HPLC method described by Wu et al., J. Agric. Food Chem. 52 (26), 7846-7856, 2004).
Protein content: 5-7% by weight compared to the dry matter of the extract (N×6.25 according to the Kjeldahl method).

### EXAMPLE 2

### Food Supplement in Capsule Form, this Composition is Particularly Suitable for Adults

Lyophilised probiotic strain, Lactobacillus rhamnosus: 35 mg, i.e. 5.10⁸ CFU;
Elder extract (Sambucus nigra) according to example 1: 50 mg;
Vitamin A: 400 μg;
Vitamin B1: 550 μg;
Vitamin B2: 700 μg;
Vitamin B5: 3 mg;
Vitamin B6: 700 μg;
Vitamin B8: 25 μg;
Vitamin B9: 100 μg;
Vitamin B12: 0.625 μg;
Vitamin C: 40 mg;
Vitamin D: 2.5 μg;
Vitamin E: 6 mg;
Iron: 2.5 mg;
Zinc: 2.5 mg;
Selenium: 15 μg;
Chromium: 12.5 μg;
Manganese: 500 μg;
Molybdenum: 12.5 μg.

### EXAMPLE 3

### Food Supplement in Sachet Form, this Composition is Particularly Suitable for Very Young Children

Lyophilised probiotic strain, Lactobacillus rhamnosus: 35 mg, i.e. 5.108 CFU;
Elder extract (Sambucus nigra) according to example 1: 25 mg;
Vitamin C: 18 mg;
Vitamin B6: 600 μg;
Vitamin E: 2.4 mg;
Zinc: 2.5 mg;
Selenium: 15 μg.

### EXAMPLE 4

### In Vitro Study of the Combined Immuno-Modulator Effects of the Strain Lactobacillus rhamnosus with an Elder Extract in Mononucleated Cells from Peripheral Blood

Material and Method

Mononucleated cells from peripheral blood are isolated from healthy subjects, re-suspended and distributed in 24 well plates at a rate of 5×10⁵ cells/wells. These cells are incubated for 18 h at 37° C. under 5% CO₂. These cells are stimulated either with 10 μg/ml of lipopolysaccharide, either in the presence of 100 μg/ml of elder extract, i.e. with 5.10⁵ Lactobacillus rhamnosus, or in the presence of 100 μg/ml of elder extract and 5.10⁵ of Lactobacillus rhamnosus. The culture supernatants are recovered to assay the different cytokines by flux cytometry, they were centrifuged at 1600 rpm for 10 minutes at 4° C. and conserved at −20° C. up to assaying.

The assaying of cytokines was carried out by the Luminex technique which is based on the principle of flux cytometry. This Luminex technology is based on the principle of ELISA assaying on 96 well microplates with, as support, microbeads incorporating two fluorochromes according to a precise ratio, which confers on them a colour code identifying them (different fluorescences). The optical system of the cytometer (Bio-Plex 200) is constituted of two lasers: a red laser (λ=635 nm) excites in each microbead the mixture of colorants that defines it, and thus identifies the cytokine to be assayed. The second green laser, (λ=532 nm) excites the reporter fluorochrome attached to the specific detection antibody in order to quantify the cytokine. The system is managed by a computer equipped with data acquisition and analysis software (Bio-Plex Manager version 4.1). After unfreezing, the supernatants were tested pure and diluted to 1/20^{th} in culture medium, using a Milliplex kit (Millipore, reference HCYTOMAG-60K-29). This comprises specific beads, detection antibodies and standards for assaying the following cytokines: IL-iβ, IL-1RA, IL-2, IL-4, IL-5, IL-6, IL-9, IL-10, IL-12p70, IL-13, IL-15, IL-17A, CCL2, CCL3, CCL4, CCL5, CCL22, CXCL8, CXCL10, TNFα, IFNγ, GM-CSF.

The concentration of each cytokine is expressed in pg/ml, it has been averaged from values obtained by 6 donors. The detection threshold of each cytokine is 3.2 pg/ml. For samples under the detection threshold, an arbitrary value corresponding to the detection threshold×½, i.e. 1.6 pg/ml has been attributed. Statistical analysis compared the log₁₀ of the concentration of each cytokine through an ANOVA.

### Results

The concentrations obtained for each condition of stimulation by cytokine are grouped together in tables 1 and 2. For there to be synergy at the level of a cytokine, it is necessary that:
the concentration of cytokine obtained experimentally by stimulating mononucleated cells from peripheral blood simultaneously with the probiotic and the elder extract, is greater than the sum of the concentrations of cytokine observed individually with elder and with the probiotic;
the median concentration of cytokine obtained with the combination for the 6 donors is significantly greater both than that obtained with elder and that obtained with the probiotic.

In table 1 are summarised the results of the activity of the elder extract combined with the probiotic Lactobacillus rhamnosus and the contribution of each of the active ingredients on the production of cytokines by mononucleated cells from human peripheral blood.

TABLE 1

| | Effect | Effect | Effect L. rhamnosus + | |
|---|---|---|---|---|
| Cytokines | elder | L. rhamnosus | elder | Synergy |
| IL-iβ | +/− | + | ++ | No |
| IL-1RA | + | + | ++++ | Yes |
| IL-2 | − | − | − | No |
| IL-4 | + | − | + | No |
| IL-5 | − | − | − | No |
| IL-6 | + | ++ | ++++ | Yes |
| IL-9 | − | − | − | No |
| IL-10 | +/− | + | ++++ | Yes |
| IL-12p70 | − | + | + | No |
| IL-13 | − | − | − | No |
| IL-15 | − | − | − | No |
| IL-17A | − | − | − | No |
| CCL2 | ++ | +++ | +++ | No |
| CCL3 | ++ | +++ | ++++ | Yes |
| CCL4 | ++ | +++ | ++++ | Yes |
| CCL5 | − | − | − | No |
| CCL22 | +/− | + | ++ | No |
| CXCL8 | ++ | +++ | ++++ | Yes |
| CXCL10 | i | ++ | + | No |
| TNFα | + | ++ | | No |
| IFNγ | − | + | + | No |
| GM-CSF | − | + | ++++ | Yes |

An increase of IL-iβ in response to the elder is only observed in 2 of the 6 donors tested. The probiotic triggers a production of IL-iβ in ⅚ of the donors.

The combination of elder and probiotic increases the production of IL-iβ, but does not reach statistical significance.

All the donors tested produce IL-IRA (antagonist to the receptor of IL-1) in response to elder, suggesting the anti-inflammatory activity of elder. All the donors tested produce IL-IRA in response to Lactobacillus rhamnosus. The concentration of IL-IRA obtained with the elder probiotic combination for the 6 donors is significantly greater than both elder and the probiotic. Furthermore the concentration of IL-IRA obtained with the elder probiotic combination is greater than the sum of that obtained by the probiotic and that obtained by elder.

There is thus synergy of the 2 active ingredients for the production of IL-1RA.

An increase of IL-2 in response to elder is only observed for a single one of the 6 donors tested. Three of the 6 donors respond to the probiotic by a moderate production of IL-2. The combination of the 2 active ingredients has no impact on the production of IL-2.

None of the donors tested responds to elder through a production of IL-5. In the same way, none of the donors tested responds to the probiotic by a production of IL-5. The combination of the 2 active ingredients has no impact on the production of IL-5.

All the donors tested produce IL-6 in response to elder. Five of the 6 donors respond to the probiotic, it should be noted that the non-responder has the highest basal rate of IL-6. For the responders, the probiotic is a more powerful inducer of IL-6 than elder. The combination of the 2 active ingredients causes a synergetic production of IL-6.

Table 2 shows an example of results of the activity of elder extract combined with the probiotic Lactobacillus rhamnosus and the contribution of each of the active ingredients on the production of IL-6 by mononucleated cells from human peripheral blood obtained from 6 donors.

TABLE 2

| | Donor | Donor | Donor | Donor | Donor | Donor |
|---|---|---|---|---|---|---|
| Groups | 1 | 2 | 3 | 4 | 5 | 6 |
| Control | 1.6 | 1.6 | 7 | 1.6 | 69 | 4 |
| Prob | 148 | 301 | 968 | 362 | 51 | 1148 |
| Elder | 17 | 27 | 44 | 12 | 405 | 17 |
| Prob + | 1258 | 1426 | 5398 | 1721 | 6081 | 5369 |
| elder | | | | | | |

Prob: probiotic, Lactobacillus rhamnosus

None of the donors tested responds to elder by a production of IL-9. Similarly, none of the donors tested responds to the probiotic by a production of IL-9. The combination of the 2 active ingredients has no impact on the production of IL-9.

Four of the 6 donors tested produce IL-10 in response to elder. All the donors tested produce IL-10 in response to the probiotic. The concentration of IL-10 obtained with elder+the probiotic for the 6 donors is significantly greater both than that obtained with elder and that obtained with the probiotic. Furthermore, the concentration of IL-10 obtained with the elder probiotic combination is greater than the sum of that obtained by the probiotic and that obtained by elder, there is thus synergy of the 2 active ingredients for the production of IL-10.

None of the donors tested responds to elder by a production of IL-12p70. On the other hand, all the donors produce IL-12p70 in response to the probiotic. The combination of elder and the probiotic does not potentialise the production of IL-12p70.

None of the donors tested responds to elder by a production of IL-15. Similarly none of the donors tested responds to the probiotic by a production of IL-15. The combination of the 2 active ingredients has no impact on the production of IL-15.

None of the donors tested responds to elder by a production of IL-17A. The same is true for the response to the probiotic: it does not induce the production of IL-17A in any of the donors. The combination of the 2 active ingredients has no impact on the production of IL-17A.

All the donors tested respond to elder by a production of CCL2. Five of the 6 donors respond to the probiotic, it should be noted that the non-responder has the highest basal rate of CCL2, the probiotic triggers a stronger response than elder. The combination of the 2 active ingredients stimulates a high secretion of CC12 significantly greater than elder but not the probiotic.

All the donors tested respond to elder by a production of CCL3. Five of the 6 donors respond to the probiotic, it should be noted that the non-responder has the highest basal rate of CCL3, the probiotic triggers a stronger response than elder. The concentration of CCL3 obtained with elder+the probiotic for the 6 donors is significantly greater both than that obtained with elder and that obtained with the probiotic. Furthermore, the concentration of CCL3 obtained with the elder probiotic combination is greater than the sum of that obtained by the probiotic and that obtained by elder, there is thus synergy of the 2 active ingredients for the production of CCL3.

All the donors tested respond to elder by a production of CCL4. All the donors tested produce CCL4 in response to the probiotic, except for one donor which has a high basal rate of CCL4, the probiotic triggers a stronger response than elder. The concentration of CCL4 obtained with elder+the probiotic for the 6 donors is significantly greater both than that obtained with elder and that obtained with the probiotic. Furthermore, the concentration of CCL4 obtained with the elder probiotic combination is greater than the sum of that obtained by the probiotic and that obtained by elder, there is thus synergy of the 2 active ingredients for the production of CCL4.

None of the donors tested responds to elder by a production of CCL5. A single one of the 6 donors produces CCL5 in response to the probiotic. The combination of elder and the probiotic has no action on CCL5.

Only 2 of the 6 donors tested produce CCL22 in response to elder. All the donors tested produce CCL22 in response to the probiotic. The combination of the 2 active ingredients has no synergetic action on CCL22.

All the donors tested produce CXCL8 in response to elder. Five of the 6 donors respond to the probiotic, it should be noted that the non-responder has the highest basal rate of CXCL8, the probiotic triggers a stronger response than elder. The combination of the 2 active ingredients causes a synergic production of CXCL8.

None of the donors tested produces CXCL10 in response to elder, conversely elder inhibits significantly the constitutive production of CXCL10. Five of the 6 donors respond to the probiotic. The combination of the 2 active ingredients shows that elder significantly inhibits the production induced by the probiotic.

All the donors tested produce TNFα in response to elder. All the donors tested produce TNFα in response to the probiotic. This production is more intense than that triggered by elder. The combination of elder and the probiotic has no impact on the production of TNFα.

None of the donors tested produces IFNγ in response to elder, on the other hand, all the donors tested produce IFNγ in response to the probiotic. The combination of elder and the probiotic has no impact on the production of IFNγ.

None of the donors tested produces GM-CSF in response to elder. On the other hand 5 of the 6 donors produce GM-CSF in response to the probiotic. The concentration of GM-CSF obtained with elder+the probiotic for the 6 donors is significantly greater both than that obtained with elder and that obtained with the probiotic. Furthermore, the concentration of GM-CSF obtained with the elder probiotic combination is greater than the sum of that obtained by the probiotic and that obtained by elder, there is thus synergy of the 2 active ingredients for the production of GM-CSF.

The lack of production of IL-2, IL-5 and IL-9, of cytokines which stimulate the proliferation of T cells, suggests that the combination should not activate the clonal expansion of T lymphocytes.

The production of IFNγ and IL-12p70, typical of a type 1 response, are due to Lactobacillus rhamnosus, whereas the modest production of IL-4, characteristic of a type 2 response, is linked to the elder extract. The type of response is estimated by the ratio IFNγ/IL-4: the response is type 1 for a ratio greater than 100, type 0 when this ratio is comprised between 0.01 and 100 and type 2 if it is less than 0.01. This ratio is 600 for Lactobacillus rhamnosus, 3 for the elder extract and 119 for the elder+Lactobacillus rhamnosus combination, which is thus type 1. The strong type 1 response induced by the probiotic is thus well counterbalanced by the production of IL-triggered by elder.

The productions of IL-iβ, IL-6, CCL2, CCL3, CCL4, CCL5, CCL22, CXCL8, TNFα and GM-CSF by the elder and Lactobacillus rhamnosus combination suggest an inflammatory activity with recruitment and activation of cells on the inflammation site. Given the chemokines involved (CCL2, CCL3, CCL4, CCL5, CCL22, CXCL8), there will be attraction preferentially of cells with innate immunity, granulocyte and monocytes/macrophages type, but also delymphocytes. This pro-inflammatory activity will be modulated by the production of IL-IRA, anti-inflammatory cytokine which links itself to the same membrane receptor as IL-1, preventing it from sending its signal to the cell. Furthermore, the inhibitor effect of elder on the constitutive and induced productions of CXCL10, chemokine attracting the activated lymphocytes, NK cells but also eosinophils, goes in the same sense. The pro-inflammatory activity of the probiotic thus seems to be counterbalanced by the anti-inflammatory effect of elder.

### CONCLUSIONS

Under the conditions tested, the inventors have shown that the combination of an elder extract and Lactobacillus rhamnosus synergises as regards the induction of IL-1RA, IL-6, IL-10, CCL3, CCL4, CXCL8, GM-CSF. This combination induces the production of IL-1β, IL-1RA, IL-4, IL-6, IL-10, IL-12p70, CCL2, CCL3, CCL4, CCL5, CCL22, CXCL8, IFNγ, TNFα and GM-CSF. This profile suggests that the elder+Lactobacillus rhamnosus combination stimulates the immune defences by recruiting monocytes, granulocytes and lymphocytes on the inflammatory site. The type 1 profile will then promote the development of cell type responses, particularly with activation of monocytes/macrophages and neutrophils, but without lymphoproliferative effect. The inflammatory reaction will be contained by the production of IL-IRA and IL-10, and by the inhibition of production of CXCL10, linked specifically to elder.

1. A composition comprising a combination of an elder extract and at least one strain of Lactobacillus rhamnosus characterised in that the elder extract comprises an anthocyanin content comprised between 0.5 and 25% by weight compared to the dry matter of the extract and a protein content comprised between 2 and 10% by weight compared to the dry matter of the extract.

2. The composition according to claim 1, characterised in that it comprises a quantity of Lactobacillus rhamnosus comprised between 1.10⁷ to 1.10¹¹ CFU/unit dose.

3. The composition according to claim 1, characterised in that it is a food, a food supplement or a medicine.

4. The composition according to claim 1, characterised in that it is a food supplement in a dosage form chosen from the group constituted of capsules, tablets to swallow, tablets to chew, effervescent tablets, pastilles, pills, powders, granules, oral solutions or suspensions and sublingual and buccal administration forms.

5. The composition according to claim 1, characterised in that it comprises between 20 mg and 200 mg of dry extract of elder per unit dose and between 1.10⁷ and 1.10¹¹ CFU of Lactobacillus rhamnosus per unit dose.

6. The composition according to claim 1, characterised in that it further comprises vitamins and/or mineral salts.

7. (canceled)

8. (canceled)

9. A method of stimulating immunity and/or strengthening the immune defenses and/or promoting an anti-infection and/or anti-inflammatory response and/or helping maintain vitality which comprises administering to a patient in need thereof a composition according to claim 1.

10. A method of treating or preventing symptoms of flu-like states which comprises administering to a patient in need thereof a composition according to claim 1.

11. The method according to claim 10, wherein the symptoms are caused by an infection by Influenza virus.

12. The composition according to claim 1, characterised in that it comprises between 20 mg and 100 mg of dry extract of elder per unit dose, and between 1.10⁷ and 1.10¹¹ CFU of Lactobacillus rhamnosus per unit dose.

13. The composition according to claim 1, characterised in that it comprises between 20 mg and 200 mg of dry extract of elder per unit dose and between 1.10⁸ and 1.10⁹ CFU of Lactobacillus rhamnosus per unit dose.
